(19) United States
(12) Patent Application Publication
(10) Pub. No.: US 2023/0103953 A1
(43) Pub. Date: Apr. 6, 2023

(54) SYSTEM AND COMPUTER IMPLEMENTED METHOD FOR CALCULATING ENERGY DOSES

(71) Applicant: TERGOS PESQUISA E ENSINO LTDA., Butantã, São Paulo-SP (BR)

(72) Inventors: Marcelo Victor Pires DE SOUSA, Butantã, São Paulo-SP (BR); Jorge Luiz Rosa MARINHO, Santa Cecília, São Paulo-SP (BR)

(21) Appl. No.: 17/768,148

(22) PCT Filed: Oct. 9, 2019
(86) PCT No.: PCT/BR2019/050440

§ 371 Date: Apr. 11, 2022

(51) Int. Cl. G01N 33/50; A01N 1/02

(52) U.S. Cl. CPC G01N 33/50; A01N 1/0226; A61K 41/17

(57) ABSTRACT

METHOD AND SYSTEM FOR CALCULATING ENERGY DOSES, PLATFORM FOR SHARING AND DISTRIBUTING ENERGY DOSES AND ENERGY DOSE

APPLICATION DEVICE The method (1000) comprises receiving (1010) parameters from an initial user; retrieving (1020) from a database (110) an initial dose based on the parameters of the initial user; adjusting (1030) the initial dose based on parameters of the initial user and a simulation of an effect of the initial dose on a standard organism, generating an adjusted initial dose; receiving (1040) feedback from the initial user regarding the effect of the adjusted initial dose; calculating (1050) a final dose based on the feedback from the initial user and the adjusted initial dose; receiving (1060) feedback from a target user regarding the effect of the final dose; calculating (1070) an adjusted final dose based on the feedback from the target user and the final dose.

### FIELD OF THE INVENTION

The present invention refers generally to a method and a system for calculating energy doses, a platform for sharing and distributing energy doses and an energy dose application device.

### BACKGROUND OF THE INVENTION

Organisms can have their functions changed by exchanging energy with the environment in which they live, through natural processes or artificially created processes. Many of these energy exchanges are known from the state of the art for therapeutic purposes.

One of the most well-known energy exchange-based therapies is photobiomodulation, formerly known as laser therapy, low-intensity light therapy etc., in which cellular biochemical reactions are stimulated by the deposition of electromagnetic energy in specific biological tissues.

Several diseases or acute or chronic clinical conditions can be treated by photobiomodulation, such as osteoarthritis, arthritis, neuralgias, low back pain, rheumatoid arthritis, and tendinopathy.

There are hundreds of patents and publications relating to therapeutic devices. However, in general, such documents usually refer to a device or method of use of that device.

It is generally found that the systems and devices of the state of the art are very operator dependent, and extensive application protocol manuals are required for applicators to perform every possible therapy.

Therefore, optimal doses for treatment are hardly achieved merely by operator experience, especially when it is considered that each organism/user may react differently to the same energy dose.

Document BR 11 2019 001573-6 discloses light therapy device controllers, light application elements, light guides, light guide arrangements configured to apply custom light therapy to one or more patients with configurations of set of components, dosage and elements of the light therapy application (for example, bandages, garments, bracelets, inserts etc.) suitable for applying light therapy to one or more areas of the body of the patient and associated tissues, as well as sensors to monitor treatment progress and dosage optimization.

However, document BR 11 2019 001573-6 does not disclose how dosage optimization is performed, that is, such a procedure is performed merely based on the experience of the operator of the therapy application element.

In turn, document PI 0709027-7 discloses devices and processes for using electromagnetic radiation and other forms of energy to treat a tissue volume at a depth, including treatments to prevent and relieve pain and promote tissue healing.

Although document PI 0709027-7 suggests mathematical simulations for the behavior of electromagnetic radiation in the biological tissues of an organism, there is no projection to systematize the creation of treatment protocols.

Thus, it would be advantageous to propose solutions to unify the explanation of the mechanism of action of non-pharmacological therapies and to systematize the creation, application and commercialization of these therapeutic processes based on energy exchanges with the organism.

### DESCRIPTION OF THE INVENTION

It is an object of the present invention to propose a method and system for calculating energy doses that enable the precise and accurate determination and quantification of how energy must be supplied to an organism in order to generate a replicable and prescriptible therapeutic response.

The method and system result in the exact protocol for generating therapeutic effects. These protocols are digitized and can therefore be distributed and accessed as information on a communication network, such as the internet.

It is an object of the present invention to propose a device for capturing the information needed for treatment and converting it into energy, which in turn is emitted by the device and absorbed by the organism.

Finally, another objective of the present invention is to propose a digital platform-type information system that allows interactions between participants to take place in a virtual environment.

One or more of the above-mentioned objectives of the invention, among others, are achieved by a method for calculating energy doses comprising the steps of: receiving a plurality of parameters of at least one initial user; retrieving from a database at least one initial dose based on at least one of the parameters of the at least one initial user; adjusting the at least one initial dose based on at least one of the parameters of the at least one initial user and a simulation of at least one effect of the initial dose on a standard organism to generate at least one adjusted initial dose; receiving feedback information from the at least one initial user regarding the effect of the at least one adjusted initial dose; calculating at least one final dose based on feedback information from the at least one initial user and the at least one adjusted initial dose; receiving feedback information from at least one target user regarding the effect of the at least one final dose; and calculating at least one adjusted final dose based on feedback information from the at least one target user and the at least one final dose.

One or more of the above-mentioned objectives of the invention, among others, are also achieved by means of an system for calculating energy doses comprising: a processor; and a database associated with the processor, wherein the processor is configured to perform the steps of: receiving a plurality of parameters from at least one initial user; retrieving from the database at least one initial dose based on at least one of the parameters of the at least one initial user; adjusting the at least one initial dose based on at least one of the parameters of the at least one initial user and a simulation of at least one effect of the initial dose on a standard organism to generate at least one adjusted initial dose; receiving feedback information from the at least one initial user regarding the at least one adjusted initial dose; calculating at least one final dose based on feedback information from the at least one initial user and the at least one adjusted initial dose; receiving feedback information from at least one target user regarding the effect of the at least one final dose; and calculating at least one adjusted final dose based on feedback information from the at least one target user and the at least one final dose.

One or more of the above-mentioned objectives of the invention, among others, are also achieved by means of a platform for sharing and distributing energy doses, comprising: a calculation module; a database; and a user interface; wherein the calculation module comprises a processor configured for: sending, by means of a communication network, at least one energy dose stored in the database to the user interface; wherein the energy dose is selected based on a plurality of parameters of a user associated with the user interface; receiving, by means of the communication network, feedback information from the user regarding the energy dose; adjusting the energy dose based on the feedback information of the user; and storing the adjusted energy dose in the database.

One or more of the above-mentioned objectives of the invention, among others, are also achieved by an energy dose application device comprising a console comprising a processor configured to control at least one emitter, the at least one emitter configured to emit energy to be absorbed by a biological tissue of an organism; the at least one emitter comprising at least one transducer and a mechanical structure for accommodating the at least one transducer; the at least one transducer comprising a printed circuit in communication with the emitter and at least one power source.

### BRIEF DESCRIPTION OF THE DRAWINGS

The objects, technical effects and advantages of the method, system, platform and device, objects of the invention, will be apparent to those skilled in the art from the following detailed description, which refers to the accompanying figures, which illustrate an exemplary, but not limiting embodiment of the invention.

FIG. 1 presents a flow chart of the method object of the present invention.

FIG. 2 presents a schematic diagram of the platform object of the present invention.

FIG. 3 presents a schematic diagram of a device according to an embodiment of the present invention.

FIG. 4 presents a graph demonstrating the pain evolution of a first target user with knee osteoarthritis by the visual analogue scale (VAS).

FIG. 5 presents a graph demonstrating the pain evolution of a second target user with knee osteoarthritis by the visual analogue scale (VAS), and the arrow indicates when the last treatment session was performed.

FIG. 6A presents a graph demonstrating the evolution of pain over 10 sessions of a target user group with knee osteoarthritis by the visual analogue scale (VAS) compared to another target user group receiving placebo sessions.

FIG. 6B presents an image of a thermographic camera showing a target user's legs before 10 photobiomodulation sessions and after 10 sessions.

FIGS. 7A to 7H present different embodiments of the device object of the present invention.

### DESCRIPTION OF EMBODIMENTS OF THE INVENTION

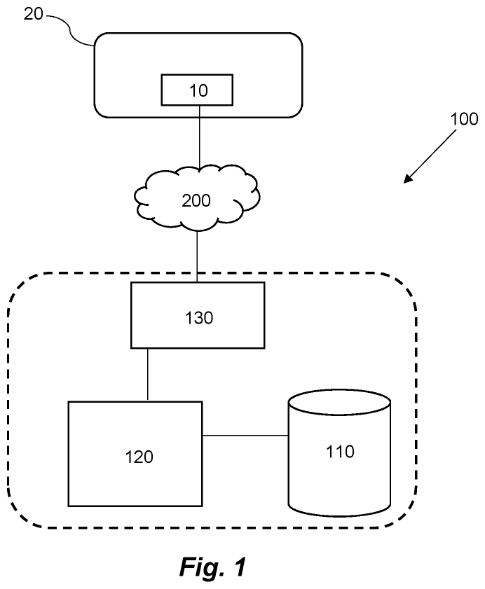

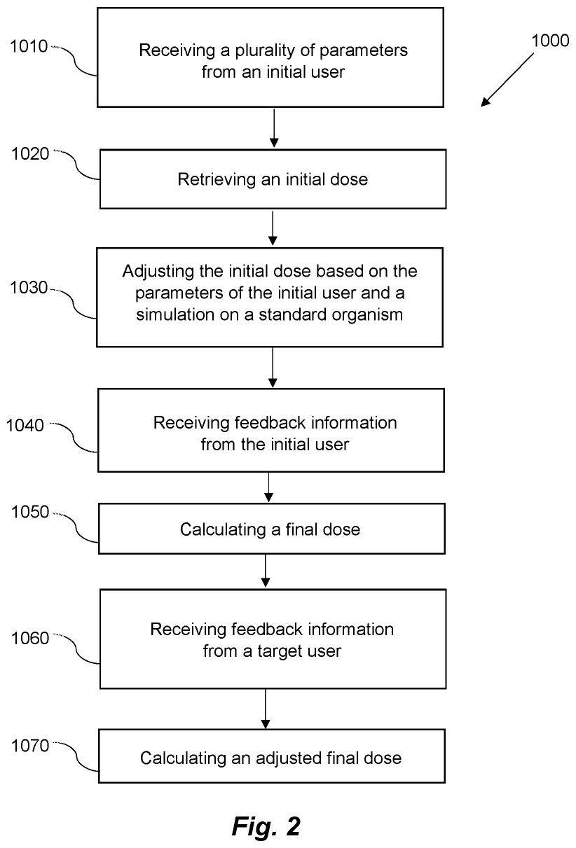

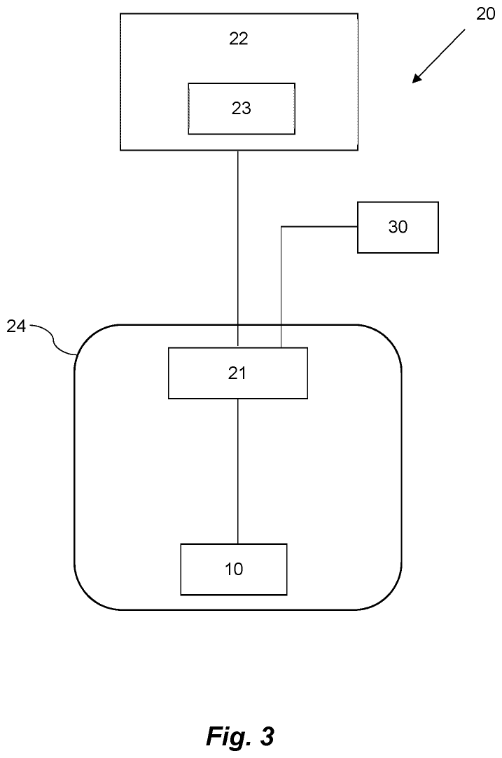

Firstly, it should be highlighted that the method, system, platform and device, objects of the invention, will be described below according to particular embodiments shown in the attached FIGS. 1 to 4B, but not limiting, since their embodiment may be carried out in different ways and variations and according to the application desired by the person skilled in the art.

The use of the term “a” or “an” in this specification does not indicate a quantity limitation, but the existence of at least one of the listed elements/components/items. The use of the term “or” indicates any or all of the listed elements/components/items. The use of the term “comprise”, “endowed”, “provided” or a similar term indicates that the element/component/item listed in front of said term is part of the invention, but it does not exclude other unlisted elements/components/items. The use of the term “associate”, “connect” or similar terms may refer to physical, mechanical, pneumatic, fluidic, hydraulic, electrical, electronic or wireless connections, either directly or indirectly.

As can be seen from FIG. 1, the method 1000 for calculating energy doses comprises a step of receiving 1010 a plurality of parameters from at least one initial user.

The parameters are selected from a group consisting of, but not limited to: condition, age, weight, height, gender, skin color, and amount of adipose tissue. In general, all of the parameters listed may influence the effect caused by electromagnetic radiation when applied to an organism.

Based on at least one parameter of the at least one initial user, the method 1000 proceeds to a step 1020 of retrieving 1020 from a database 110 at least one initial dose.

The initial dose can be interpreted as a treatment protocol comprising the energy parameters to be applied to the initial user. A non-limiting denomination of these treatment protocols is “digital medicine” (DM) in the sense that information regarding the treatment protocol can be converted into computer readable instructions and stored on a computer readable medium.

In one embodiment, the energy to be applied to the initial user is electromagnetic energy, wherein its parameters include type of equipment used, radiation wavelength, power per unit of area, and exposure time of the organism to radiation.

However, a person skilled in the art will immediately appreciate that other forms of energy may be used for the same purpose, such as mechanical energy from vibration, thermal energy, among others.

In one embodiment of the invention, initial dose values are continuously retrieved from the specialized literature and stored in the database 110. Thus, the collected data help to assess which initial parameters are most likely to converge to digital medicines approved.

Continuing, the method 1000 comprises a step of adjusting 1030 the at least one initial dose based on at least one of the parameters of the at least one initial user and a simulation of at least one effect of the initial dose on a standard organism to generate at least one adjusted initial dose.

Simulating the effect of the initial dose on a standard organism generally comprises steps of modeling the behavior of electromagnetic radiation in the organism (photokinetics) and simulating its therapeutic effect (photodynamics).

In one embodiment of the present invention, the step of adjusting the at least one initial dose comprises: determining a power per unit of area E from the at least one initial dose; simulating the at least one effect of the initial dose from the power per unit of area E on at least one biological tissue m of the standard organism for a predetermined exposure time period t.

In particular, the at least one effect of the initial dose comprises the variation in concentration of at least one biological marker n after exposure of the at least one biological tissue m to power per unit of area E for the predetermined time period t; and adjusting the at least one initial dose based on the simulated variation in concentration of the at least one biological marker n and at least one of the parameters of the at least one initial user.

If the energy applied to the initial user is electromagnetic energy, the power per unit of area E may be referred to as irradiance.

From the known irradiance on the surface of the organism E, the Photon Transport Equation (PTE) is applied, considering now that the surface of the biological tissue has a radiance given by L₀=E and considering the edge effect, air/biotissue with reflection interface, transmission and refraction of photons, according to the following equation:

$$\frac{\partial L ( \vec{r} , \hat{s} , t ) / c}{\partial t} = - \hat{s} \cdot ∇ L ( \vec{r} , \hat{s} , t ) - {μ}_{t} L ( \vec{r} , \hat{s} , t ) + {μ}_{s} {\int }_{4 π} L ( \vec{r} , {\hat{s}}^{′} , t ) P ( {\hat{s}}^{′} , \hat{s} ) d {Ω}^{′} + S ( \vec{r} , \hat{s} , t )$$

wherein:

c is the speed of light in the tissue, as determined by the relative refractive index;

μₜ=μₐ+μₛ is the extinction coefficient;

P (ŝ′, ŝ) is the phase function, representing the probability of light with the propagation direction ŝ′ being scattered at a solid angle dΩ around ŝ. In most cases, the phase function depends only on the angle between the directions of scattering ŝ′ and incident ŝ, that is, P (ŝ′, ŝ)=P (ŝ′·ŝ). Scattering anisotropy can be expressed as:

$$g = {\int }_{4 π} ( {\hat{s}}^{′} \cdot \hat{s} ) P ( {\hat{s}}^{′} \cdot \hat{s} ) d Ω$$

and

S(\vec{r}, ŝ, t) describes the light source.

In this phase, modeling and simulation are performed considering a standard organism, or, more specifically, a “computational standard human”, which has the previously determined optical parameters of biological tissues.

The results generated from the modeling can be stored in database 110, since the photokinetic data of each biological tissue can be useful for obtaining other digital medicines.

By way of non-limiting example, simulations of the interaction of light with biological tissues such as skin, muscle and bones performed for the digital medicine of knee osteoarthritis can be reused for the digital medicine of knee ligament regeneration.

Continuing, simulating the variation in the concentration of the at least one biological marker n comprises calculating a stimulus Vₘ received from the at least one biological tissue m during the predetermined exposure time t.

More specifically, the stimulus given by Vₘ comprises the volumetric power density integrated over the predetermined time period and integrated throughout the volume of biological tissue m; for the at least one biological tissue m, that is:

Vₘ=∫∫₀^{t}EdΩdt.

Next, method 1000 comprises a step of determining the solution of the matrix equation C=M·V (Sousa's equation), wherein:

$$C = ( \begin{matrix}{C}_{1} \\ ⋮ \\ {C}_{n}\end{matrix} ) ,$$

wherein Cₙ is the variation in the concentration of the at least one biological marker n;

$$M = ( \begin{matrix}{M}_{11} & … & {M}_{1 m} \\ ⋮ & ⋱ & ⋮ \\ {M}_{n 1} & … & {M}_{nm}\end{matrix} )$$

where Mₙₘ is the susceptibility of the at least one biological tissue m to modulate the production of the at least one biological marker n, wherein the susceptibility can be any number between −1 and 1, wherein if it is −1, there is complete inhibition of production of the biological marker n; if it is 0, there is no influence, and if it is 1, there is maximum stimulation of production of the biological marker n; and

$$V = ( \begin{matrix}{V}_{1} \\ ⋮ \\ {V}_{m}\end{matrix} )$$

wherein V=(V₁ . . . Vₘ) are the stimuli received in each biological tissue m from the standard organism during the predetermined exposure time t.

The Sousa's equation shows that for each stimulus Vₘ received by a user, a series of biochemical alterations is generated, which results in variations in the concentration of the biological markers n. Among these biological markers, some will have therapeutic effect.

Then, each variation of biological marker n by tissue Cₙ is summed to determine the total variation ΔSₙ of each biological marker n in the standard organism.

If the total variations ΔSₙ do not meet a predetermined limit, the variation in the concentration is re-simulated with a new value of power per unit of area E.

On the other hand, if the variations ΔSₙ meet the predetermined limit, the at least one initial dose is adjusted based on the at least one parameter of the initial user to achieve the same values of ΔSₙ.

Similar to photokinetics, the results of the aforementioned simulations can be stored in database 110, as the photodynamic data collected from each biological tissue may be useful for obtaining other digital medicines.

Without wishing to be bound by any particular theory, the changes in a given biological tissue will be the same whenever the stimulus is the same, so that once such effects are observed, it is possible to store them in the database 110 and subsequently apply them in the development of other digital medicines.

In one embodiment of the present invention, biological markers are selected from a group comprising: IL-1β, IL-6, IL-10, TNFα; prostaglandin E2 (PGE2); metalloproteinase, PCR, CPK, VHS, creatinine, urea, serotonin, dopamine; leukocytes; erythrocytes, haematocrits, hemoglobin, VCM, HCM, CHCM, RDW, erythroblasts, leukocytes, myelocytes, metamyelocytes, rods, segmented, total neutrophils, eosinophils, basophils, lymphocytes, atypical lymphocytes, monocytes, plasmocytes, platelets; albumin, potassium, VHS, C-reactive protein and total proteins and fractions, rheumatoid factor, anti-CCP, FAN, calcium, phosphorus, vitamin D and PTH.

Following the step of adjusting the initial dose 1030, the method 1000 comprises the step of receiving feedback information from the at least one initial user regarding the effect of the at least one adjusted initial dose.

In one embodiment of the present invention, the feedback information of the initial and target users includes at least one of: results of clinical and diagnostic examinations to measure, directly or indirectly, the actual variation in the concentration of biological markers.

In addition, the results of questionnaires, examinations, and diagnoses are selected from results of a group comprising: Knee injury and Osteoarthritis Outcome Score (KOOS), World Health Organization Quality of Life-Bref (WHOQOL-bref), Leeds Assessment of Neuropathic Symptoms and Signs (LANSS); Visual Analogue Scale (VAS), thermography; cirtometry, goniometry; Sit-to-Stand (STS) test and timed up and go (TUG) test; complete blood count; synovianalysis; X-ray and nuclear magnetic resonance.

Upon receipt of feedback information, the method 1000 proceeds to a step of calculating 1050 at least one final dose based on the feedback information from the at least one initial user and the at least one adjusted initial dose.

Next, method 1000 comprises a step of receiving 1060 feedback information from at least one target user, that is, the user to be effectively treated, regarding the effect of the at least one final dose.

Finally, method 1000 comprises a step of calculating 1070 at least one adjusted final dose based on feedback information from the at least one target user and the at least one final dose.

In one embodiment, the steps of adjusting 1030 the at least one initial dose, calculating 1050 the at least one final dose, and calculating 1070 the at least one adjusted final dose are performed by distinct algorithms.

In another embodiment of the present invention, the at least one initial user is a plurality of initial users, wherein the at least one final dose comprises a set of final doses, each final dose being associated with a respective initial user, the set of final doses being stored in the database 110.

Thus, method 1000 may comprise a step of selecting a final dose from the set of final doses most compatible with the parameters of the target user.

It is particularly advantageous if the adjusted final dose is added to the set of final doses so that knowledge about other cases, clinical conditions and phenotypes of target users is acquired. In other words, the final result is constant updating and optimization of digital medicines.

In one embodiment of the present invention, the method 1000 further comprises a step of sending the adjusted final dose to a distribution platform 100 by means of a communication network 200, such as the internet.

The distribution platform 100 comprises: a calculation module 120, the database 110 and a user interface 10.

Particularly, the calculation module 120 comprises a processor configured for:

sending, by means of the communication network 200, the at least one final energy dose stored in the database 110 to the user interface 10, wherein the final energy dose is selected based on the plurality of parameters of the target user associated with the user interface 10;

receiving, by means of the communication network 200, feedback information from the target user regarding the final energy dose;

adjusting the final energy dose based on the feedback information of the target user; and

storing the adjusted final energy dose in the database 110.

Additionally, platform 100 may comprise a user access and authentication module 130 configured to control communication between the device 20 and the calculation module 120.

In one embodiment of the present invention, the user interface 10 is a component of an energy dose application device 20, more particularly a photobiomodulation therapy device.

In particular, the dose application device 20 may be determined based on the power per unit of area E.

In general, device 20 comprises: a console (21) comprising a control unit configured to control at least one emitter 22, the at least one emitter 22 configured to emit energy to be absorbed by a biological tissue of the initial user or the target user; the at least one emitter 22 comprising at least one transducer 23 and a mechanical structure for accommodating the at least one transducer 23; the at least one transducer 23 comprising a printed circuit in communication with the emitter 22 and at least one power source.

In one embodiment, the console 21 comprises the user interface 10, the user interface 10 in communication with the control unit, wherein a housing 24 encapsulates the console 21 and the user interface 10.

In general, the user interface 10 is configured to send visual, audible, and/or tactile signals and information from the console 21 to the user.

Alternatively, the console 21 may additionally be connected to a user device (or gadget) 30 configured to control the operations of device 20 through an application. The connection can be wired or wireless, for example by means of internet or Bluetooth.

Briefly, the console 21 is a computer device that sends an electrical signal to the energy emitter 22 to emit an energy dose, for example electromagnetic energy, to one or more target users.

In one embodiment of the present invention, the console 21 further comprises a data-receiving module and digital commands from the user device 30.

In a non-limiting example, data is received via Bluetooth. However, a person skilled in the art will immediately appreciate that various other forms of wireless or wired data reception can be implemented, such as via USB, Wi-Fi, 4G, 5G etc.

Additionally, the console 21 may be configured to establish communication with the user device 30.

Also, the console 21 can be configured to recognize, control and power the emitters 22 connected to it. The connection between console 21 and emitters 22 may be, for example, a multi-channel mechanical and electrical connection.

In one embodiment, recognition of emitters 22 may be accomplished by an electronic signal test, wherein each emitter 22 is configured with a specific response.

In one embodiment of the invention, the console 21 is pre-configured to control and power up to three emitters 22 simultaneously.

Self-test. It enables to know if there is any electronic fault in the console, or one of the emitters, or one of the transducers.

Thus, console 21 is configured for both converting commands received in digital signal format from the user device 30 to commands in electrical signal format, which are sent to emitters 22 and command the emission of the energy dose personalized for the target user.

Preferably, the console 21 receives electrical energy from a power source, such as a battery, and distributes it to the other components of the device 20, such as emitters 22 and transducers 23.

Advantageously, the at least one emitter 22 may be anatomically adapted to an application region on the initial user or the target user.

In short, the at least one emitter 22 is the device that emits the digital medicine, that is, the energy that, when absorbed into the biological tissues of the target user, will result in the transduction of energy into medicine generated in the organism (TERGO).

In one embodiment of the present invention, the at least one emitter 22 comprises a set of transducer 23, a mechanical structure for encapsulating and supporting the transducers 23, a power cable, a multi-channel cable for communication with the console 21; a mechanical and electrical connector with the console 21, an interface structure between the biological tissue and the transducers 23 and connection means with the transducers 23.

Preferably, the emitters 22 are configured to receive electrical signals from the console 21 and conduct them to the transducers 23.

In another embodiment of the present invention, the shape of the emitters 22 and their holding means of the transducers 23 are configured to allow the best fit for each anatomy, physiology, pathology, clinical condition, among others, of the region of the target user to receive the digital medicine.

Continuing, the transducers 23 are the devices that transduce (change the form of the energy) the electrical current received by the emitters 22 to the form of therapeutic energy that will be emitted.

In one embodiment, the transducers 23 are configured to convert electrical current to photonic energy (light with well-defined emission spectrum).

A transducer 23 comprises a printed circuit, power sources and a mechanical structure that defines the mechanical characteristics to the transducers.

Advantageously, the transducers 23 can be assembled in a variety of ways on emitters 22 that meet the anatomical, physiological and pathological needs of the target users.

Preferably, various arrangements of emitters 22 are connected to the console 21, allowing for wide range of therapeutic variations.

The transducer is the basic unit of TERGO devices. They can be associated in the most different forms to produce emitters with different application forms that are suitable for the most different anatomies, physiologies, pathologies and therapeutic procedures.

Further, the at least one power source of the at least one transducer 23 being a plurality of light-emitting diodes (LEDs), OLEDs, lasers or a combination thereof.

However, other forms of energy conversion may also be considered by a person skilled in the art such as those that convert electrical energy to pressure wave, ultrasound, electromagnetic fields, plasma etc.

A particularly advantageous configuration for LEDs is an irradiated area of 120 cm², power between 0.1 and 8 W, pulse frequency between 0 and 100 Hz and monochrome (or narrow spectrum) wavelength between 400 and 1000 nm.

Another aspect of the present invention relates to a system for calculating energy doses comprising a processor and a database 110 associated with the processor.

In a preferred embodiment, the processor is configured to perform steps 1010, 1020, 1030, 1040, 1050, 1060, and 1070, previously detailed.

Additionally, the processor may be configured to send the at least one adjusted initial dose and the at least one final dose to an energy dose application device 20 by means of a wired or wireless communication network 200, such as the internet or Bluetooth.

Furthermore, the processor may be configured to receive feedback information from the at least one initial user and the at least one target user from the energy dose application device 20 by means of the communication network 200.

Below are different examples of embodiments of emitters 22. It is worth noting that a person skilled in the art will immediately notice that the shape, dimensions and number of transducers may vary depending upon the desired application.

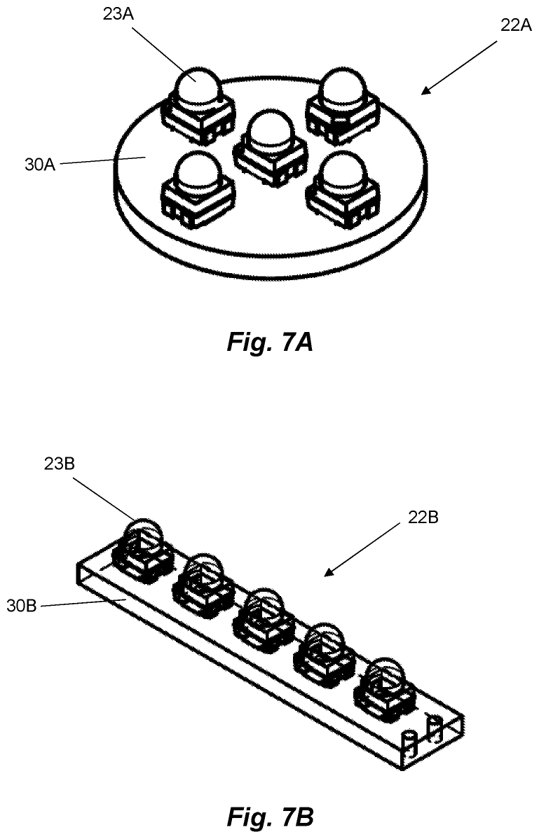

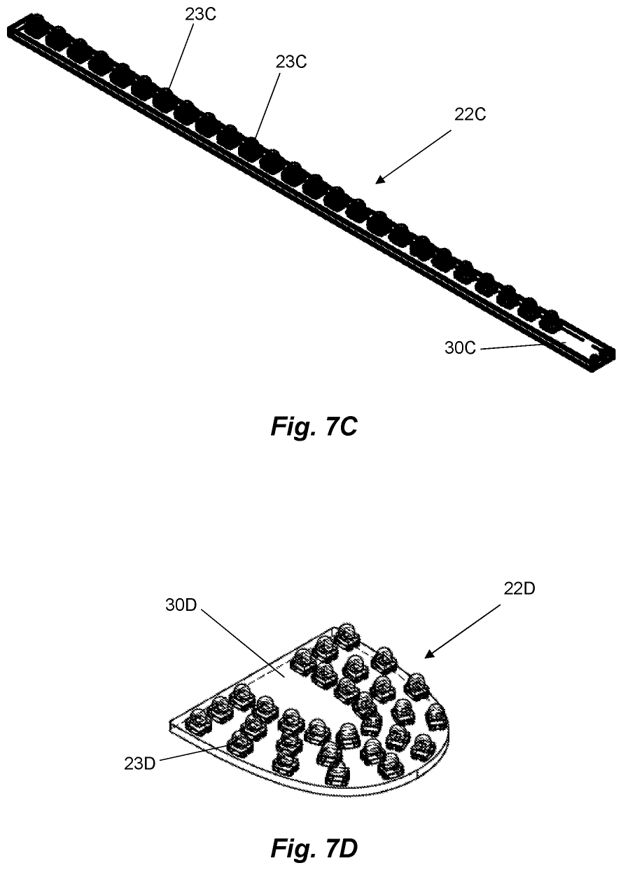

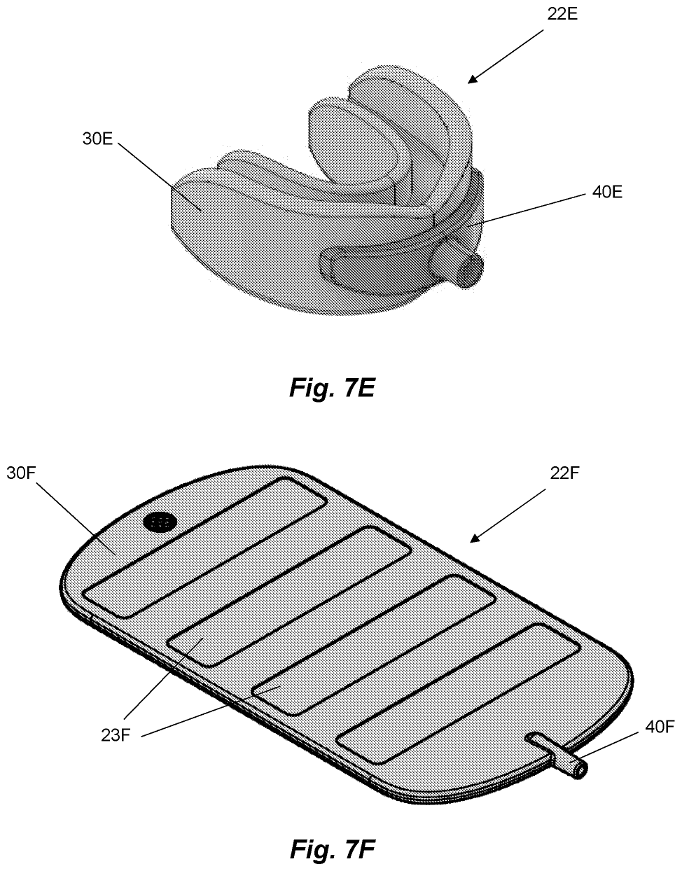

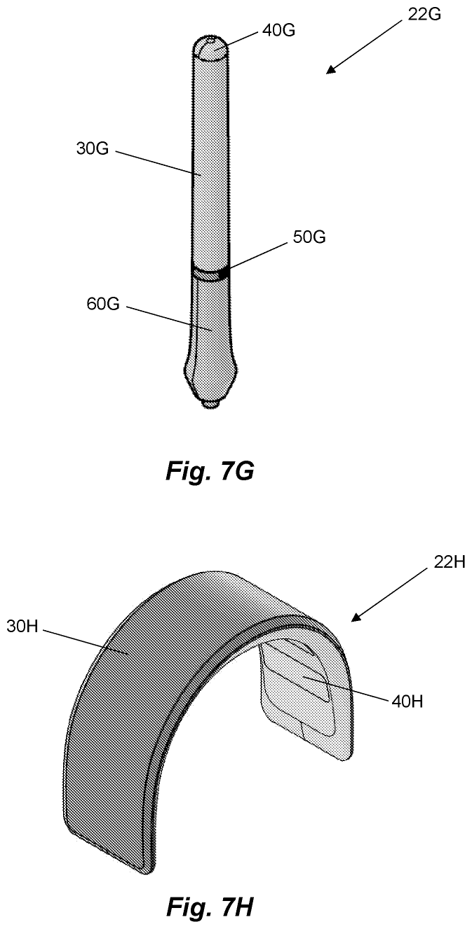

FIG. 7A shows a substantially circular shaped emitter 22A comprising 5 transducers 23A, preferably LEDs, mounted on a cylindrical shaped mechanical structure 30A.

FIG. 7B shows a substantially rectangular (linear) shaped emitter 22B comprising 5 transducers 23B, preferably LEDs, mounted on a parallelepipedal shaped mechanical structure 30B.

FIG. 7C shows a substantially rectangular (linear) shaped emitter 22C comprising 25 transducers 23C, preferably LEDs, mounted on a parallelepipedal shaped mechanical structure 30C.

Alternatively, the emitters 22 may have shapes specially adapted to the anatomy of the target user, as described below.

FIG. 7D shows a first embodiment of a buccal emitter 22D comprising a plurality of transducers 23D, preferably LEDs, mounted on a substantially semicircular shaped mechanical structure 30D.

Optionally, the buccal emitter 22D may be wrapped in a silicone structure (not shown) and it may comprise the plurality of LEDs mounted on a printed circuit board (not shown) disposed between the mechanical structure 30D and said plurality of LEDs.

FIG. 7E shows a second embodiment of a buccal emitter 22E. In particular, the buccal emitter 22E comprises a plurality of transducers (not shown) mounted on a flexible printed circuit board.

Optionally, the buccal emitter 22E comprises a silicone mechanical structure 30E that surrounds the plurality of transducers and comprises an adapter 40E for connecting a power/control cable (not shown) from the console 21.

FIG. 7F shows a pulmonary emitter 22F comprising a plurality of transducers 23F enclosed by a silicone mechanical structure 30F configured to protect the transducers 23F. Additionally, the pulmonary emitter 22F comprises an adapter 40F for connecting a power/control cable (not shown) from the console 21.

FIG. 7G shows a vaginal emitter 22G comprising a plurality of transducers (not shown) enclosed by a substantially cylindrically shaped mechanical structure 30G.

Additionally, the vaginal emitter 22G comprises a protective lens 40G, a protective ring 50G and a cover 60G. In one embodiment of the present invention, the mechanical structure 30G may be additionally enclosed by an inflatable device (not shown).

Finally, FIG. 7H shows a cranial emitter 22H comprising a plurality of transducers (not shown) enclosed by a substantially semicircular shaped mechanical structure 30H.

Optionally, the cranial emitter 22H may comprise a protective lens 40H mounted on an internal surface of the mechanical structure 30H for protection of said plurality of transducers.

Continuing, additional details and embodiments of the digital medicine distribution platform 100 will be provided.

In one embodiment of the present invention, the distribution platform 100 is a digital platform comprising 4 structures and 4 players.

More specifically, the structures are Marketspace, applications, application devices 20, and therapeutic environment, while players (or person) are Digital Medicine (DM) creators, DM prescribers, DM applicator therapists, and target users (patients) who receive one or more DM.

The digital medicine marketspace is a virtual environment in which the players of the distribution platform 100 relate. By way of non-limiting example:

creators make their DMs available by uploading to platform 100;

prescribers find the right medicines for their patients;

therapists may find therapeutic environments (usually associated with one or more prescribers) to provide their services; download the digital medicine and connect to energy dose application devices; and

patients find therapists, physicians, therapeutic environments that meet their needs, and they can follow all their therapeutic data from current and previous DM applications and receive indications of new DM.

Marketspace enables two-way commerce wherein the players can either sell their services or digital products or buy products offered by both the platform and other players.

Thus, the platform 100 can be advantageously configured to provide sellers players with a series of digital products such as: online Marketplaces, social networks, store design, Marketing, Sales and conversion, gaming, dynamic pricing, product package generation, Transaction data analysis, promotion and discount strategies, Customer Service, Finance, Productivity, Business Intelligence Reports, among others.

In one embodiment, the platform 100 can be configured to perform curation of the digital medicines in the marketspace. One of the most relevant aspects is the classification of digital medicines by profession, specialty, therapeutic environment, and level of training in TERGO creating access levels so that only specific prescribers and therapists can access the Digital Medicines of greater complexity or risk.

Applications are the interface of the distribution platform 100 with different players. Each player has specific needs in relation to the other players and the distribution platform 100, so the interfaces can be advantageously customized for each type of player.

In addition, applications connect players to platform 100 and to each other through the communication network 200, enabling a flow of information that allows clinical, professional, financial, and commercial interactions of the digital medicines.

In addition, applications allow players to access: historical data on digital medicine use; (non-confidential and/or unstructured) patient data; quantitative and qualitative data as well as evaluations and classification of digital medicines, professionals, services, therapeutic environments, among others; monitoring of player behavior on the distribution platform 100.

In one embodiment, devices 20 receive digital medicines as machine language information packets (bits) and convert them in the firmware to electrical current standards that are again converted in the transducers 23 to energy emitted to the organism, which will generate biochemical reactions with therapeutic potential.

Finally, therapeutic environments may comprise environments such as hospital beds, clinics, doctor's offices and the residence of the target user.

Turning now to the detail of the players, it is noticed that they are roles played by people within the distribution platform 100.

One person can play multiple players in different situations. By way of example, a medical scientist can create a DM, prescribe a DM (created by himself/herself or others), can perform the application of DM (playing the role of therapist) and also be patient.

The creators of Digital Medicines are natural or legal person working alone or in groups, who use the digital tools and services provided by the platform 100 to create new Digital Medicines.

Prescribers are health professionals properly trained to prescribe customized and personalized digital medicine treatments.

In addition, prescribers evaluate, examine, and diagnose the patient before, during, and after treatment. It is the prescriber who chooses the DMs and the amounts, number of applications and duration of therapy. The prescribers accompany their patients with telemedicine solutions even when they are not in the same location.

Therapists are the professionals who effectively apply the Digital Medicines. They handle the application devices 20 in accordance with practices and regulations associated with TERGO therapy and they have face-to-face contact with patients during applications.

Patients, in turn, have access to their own history, reports of their own evolution, and structured data about the DM, professionals, services, and therapeutic environments.

Thus, patients can choose professionals and clinics taking into account their needs such as those related to location, health care plans, hours etc.

Players relate to each other and to the distribution platform 100 within structures in countless ways. Some of these interactions are described in Table 1:

TABLE 1

| | | | | Therapeutic |
|---|---|---|---|---|
| | Marketspace | Applications | Devices | environment |
| Creators | Sell DM | See Dashboard with their DM data | Develop DM | Qualitative |
| | | | for devices | data generation |
| Prescribers | Buy DM | Select, customize, personalize, | Receive on | Telemedicine |
| | | prescribe treatment with | consignment | |
| | | appropriate DMs. | | |
| | | History of all treatments of their | | |
| | | patients. | | |
| Therapists | Download DM | Apply DM according to the patient's | Uses in patients | Apply DM |
| | | momentary situation. | of the prescriber | to patients |
| Patients | Player Information | Individual history of DM treatments. | See | Receive treatment |

When a digital medicine goes through the method 1000 and is ready to be emitted by the therapeutic devices 20, it is sent to the distribution platform 100, which allows digitizing various scientific, clinical, therapeutic, commercial and business aspects, such as: teaching-learning, qualification, specialization, customization, personalization, dynamic monitoring of therapy evolution, choice of products, services, professionals, therapeutic environments, relationships, channels, distribution, payments etc.

In one embodiment of the present invention, the path of the digital medicine on platform 100 comprises the following steps: upload by creator; exhibition on virtual shelves of Marketspace; purchase by prescribers, wherein the digital medicine becomes part of a “shelf of the virtual office”; therapist's access (bound and authorized by the prescriber) through his/her user device 30 to the DM to apply to the patient also bound that prescriber.

Preferably, the DM is sent from the therapist's user device 30 to the application device 20 that will be used in the treatment.

In the application device 20, the digital medicine information is converted into energy emission in the appropriate parameters. In the organism of the patient, there is the conversion of this energy into endogenous medicines.

When DM is applied, it also connects to a user device 30 of the patient so that it can provide effectiveness data over time, generating feedback information for DM optimization and generation of scientific, clinical, business, commercial intelligence etc.

Advantageously, the present invention demonstrates how to create the therapeutic process itself, that is, which Energy Transduction process will result in a Medicine Generated by the Organism. To this end, the current logic of creating an apparatus and demonstrating that such an apparatus has various therapeutic uses is reversed in the present invention.

The method associated with the present invention leads to discover Medicines Generated by the Organism and only then to develop or select the most suitable devices to cause the therapeutic effects previously observed.

Digital distribution platform 100 creates a virtual environment for interactions between people, organizations and institutions. Thus, it becomes possible to create, collaborate, treat, negotiate, market etc. in a digital environment.

The therapy created, applied and negotiated with the methods, devices and systems described herein is reduced to information and, therefore, it can be digitized and converted into machine language. Consequently, the treatment protocol or energy doses may be referred to as “digital medicine” (DM).

In general, the present invention should not be compared to:

1) specific medical devices of the state of the art, since the combination of transducers 23, emitters 22 and consoles 21, as well as their digital connectivity, allow the creation of numerous application devices 20;

2) a single medicament, remedy or therapeutic procedure known from the state of the art, since the method 1000 disclosed by the present invention allows to discover ways to induce organisms (humans, animals, plants and other living beings) to produce substances, making the organism function as a “biological medicine factory”. Thus, the present invention is not reducible to a single medicine, since it can induce organisms to manufacture medicines of the most varied types;

3) mere quantification of an energy dose absorbed in the organism, since this calculation alone is not sufficient to discover which biological markers will be produced and/or consumed by the organism to generate therapeutic effect.

The present invention describes how to make digital medicine production and it leads to significant scientific, clinical, technological and commercial advantages over the production of pharmacological medicine. Advantageously, the production of Digital Medicines (DM) allows the efforts of research, development and clinical validation of a new drug to be exchanged for computational simulations of the interaction of energy with matter and its biological and therapeutic results.

### Example 1—Knee Osteoarthritis

Diagnostic hypothesis: Patient diagnosed by medical physical examination.

Imaging examinations: X-ray and/or magnetic resonance (Confirmation of medical diagnosis).

Treatment steps:

1^{st} Step—Realization and evaluation of the complete blood count, determination of inflammatory markers and synovianalysis before the beginning of treatment;

2^{nd} Step—Treatment with TERGO-based digital medicine with the number of sessions recommended by the healthcare professional;

3^{rd} Step—Realization and evaluation of the complete blood count, inflammatory markers and synovianalysis after the last treatment session; and

4th Step—Evaluation of imaging examinations 6 months after the last treatment session.

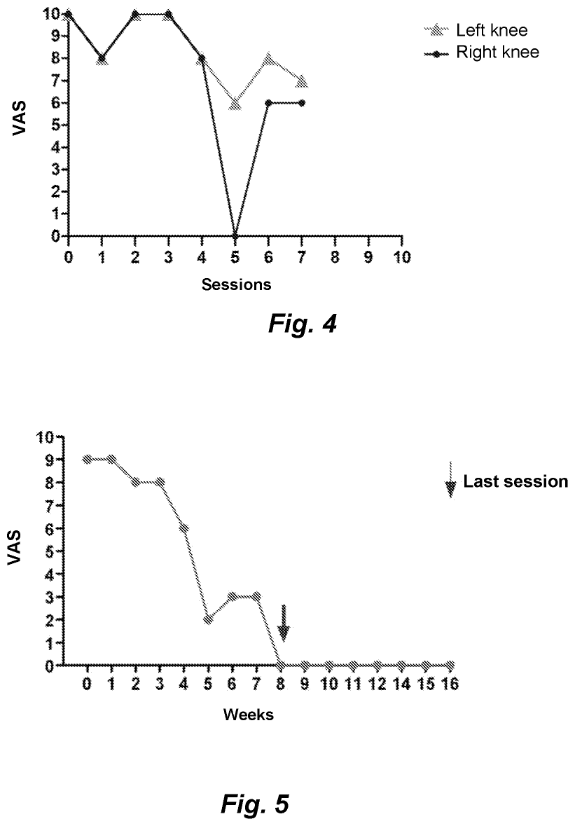

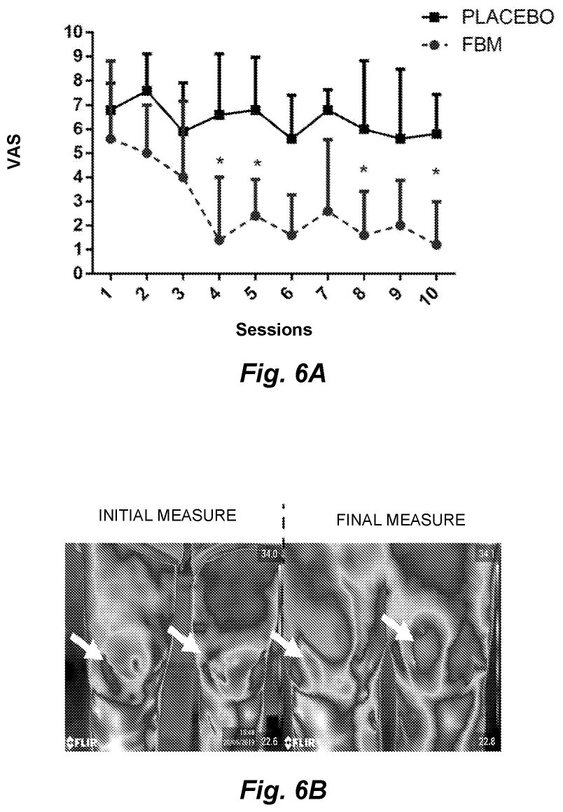

Satisfactory results of the therapy are illustrated by FIGS. 4A and 4B, wherein it is verified that the patient's pain level measured by a visual analogue scale (VAS) decreased consistently over the therapy sessions.

Although the description of the particular embodiment above refers to a particular example, the present invention may be embodied in analogous ways and it may have modifications in its implementation form, so that the scope of protection of the invention is limited only by the content of the appended claims, including all possible equivalent variations linked to the method, system, platform and device.

1. A COMPUTER IMPLEMENTED METHOD (1000) FOR CALCULATING ENERGY DOSES, comprising the steps of:
receiving (1010), by a processor, a plurality of parameters from at least one initial user;
retrieving (1020) from a database (110), by the processor, at least one initial dose based on at least one of the parameters of the at least one initial user;
adjusting (1030), by the processor, the at least one initial dose based on at least one of the parameters of the at least one initial user and a simulation of at least one effect of the initial dose on a standard human organism to generate at least one adjusted initial dose;
receiving (1040), by the processor, feedback information from the at least one initial user regarding the effect of the at least one adjusted initial dose;
calculating (1050), by the processor, at least one final dose based on the feedback information from the at least one initial user and the at least one adjusted initial dose;
receiving (1060), by the processor, feedback information from at least one target user regarding the effect of the at least one final dose; and
calculating (1070), by the processor, at least one adjusted final dose based on the feedback information from the at least one target user and the at least one final dose;
wherein the at least one initial dose, the at least one adjusted initial dose, the at least one final dose and the at least one adjusted final dose comprise electromagnetic energy with a wavelength between 400 to 1000 nm;
wherein the step of adjusting (1030) the at least one initial dose comprises:
determining a power per unit of area E from the at least one initial dose;
simulating the at least one effect of the initial dose from power per unit of area E on at least one biological tissue m of the standard human organism for a redetermined exposure time period t;
wherein the at least one effect of the initial dose comprises the variation in concentration of at least one biological marker n after exposure of the at least one biological tissue m to power per unit of area E for the predetermined time period t and
adjusting the at least one initial dose based on the simulated variation in concentration of the at least one biological marker n and at least one of the parameters of the at least one initial user, and
wherein the simulating variation in the concentration of the at least one biological marker n comprises calculating a stimulus Vₘ received from the at least one biological tissue m during the predetermined exposure time t;
wherein the stimulus Vₘ comprises the volumetric energy density integrated over the predetermined period of time for the at least one biological tissue m, that is,
Vₘ=∫∫₀^{t}EdΩdt;
determining the solution of the matrix equation C=M·V, wherein
$$C = ( \begin{matrix}{C}_{1} \\ ⋮ \\ {C}_{n}\end{matrix} ) ,$$
wherein Cₙ is the variation in the concentration of the at least one biological marker
$$M = ( \begin{matrix}{M}_{11} & … & {M}_{1 m} \\ ⋮ & ⋱ & ⋮ \\ {M}_{n 1} & … & {M}_{nm}\end{matrix} )$$
wherein Mₙₘ is the susceptibility of the at least one biological tissue m to modulate the production of the at least one biological marker n; wherein the susceptibility can be any number between −1 and 1, wherein if it is −1, there is complete inhibition of production of the biological marker n; if it is 0, there is no influence, and if it is 1, there is maximum stimulation of production of the biological marker n; and
$$V = ( \begin{matrix}{V}_{1} \\ ⋮ \\ {V}_{m}\end{matrix} )$$
wherein V=(V₁ . . . Vₘ) are the stimuli received in each biological tissue m from the standard human organism during the predetermined exposure time t; and
summing each variation of biological marker n by tissue Cₙ to determine the total variation ΔSₙ of each biological marker n in the standard human organism.

2.-4. (canceled)

5. The METHOD (1000) according to claim 1, further comprising the steps of:
re-simulating the variation in the concentration with a new value of power per unit of area E if the total variations ΔSₙ do not meet a predetermined limit; and
adjusting the at least one initial dose based on the at least one parameter of the initial user to achieve the same values of ΔSₙ if variations ΔSₙ meet the predetermined limit.

6. The METHOD (1000) according to claim 1, wherein the feedback information from the at least one initial user and the feedback information from the at least one target user includes at least one of: results of clinical and diagnostic examinations to measure, directly or indirectly, actual variation in the concentration of biological markers.

7. The METHOD (1000) according to claim 6, wherein the results of questionnaires, examinations and diagnoses are selected from the results of a group consisting of: Knee injury and Osteoarthritis Outcome Score (KOOS), World Health Organization Quality of Life-bref (WHOQOL-bref), Leeds Assessment of Neuropathic Symptoms and Signs (LANSS); Visual Analogue Scale (VAS), thermography; cirtometry, goniometry; Sit-to-Stand (STS) test and timed up and go (TUG) test; complete blood count; synovianalysis; X-ray and nuclear magnetic resonance.

8. The METHOD (1000) according to claim 1, wherein the at least one biological marker n is markers are selected from a group comprising: IL-1β, IL-6, IL-10, TNFα; prostaglandin E2 (PGE2); metalloproteinase, PCR, CPK, VHS, creatinine, urea, serotonin, dopamine; leukocytes; erythrocytes, haematocrits, hemoglobin, VCM, HCM, CHCM, RDW, erythroblasts, leukocytes, myelocytes, metamyelocytes, rods, segmented, total neutrophils, eosinophils, basophils, lymphocytes, atypical lymphocytes, monocytes, plasmocytes, platelets; albumin, potassium, VHS, C-reactive protein and total proteins and fractions, rheumatoid factor, anti-CCP, FAN, calcium, phosphorus, vitamin D, and PTH.

9. The METHOD (1000) according to claim 1, wherein the plurality of user parameters are selected from a group consisting of: condition, age, weight, height, gender, skin color and amount of adipose tissue.

10. The METHOD (1000) according to claim 1, wherein the steps of adjusting (1030) the at least one initial dose, calculating the at least one final dose (1050), and calculating (1070) the at least one adjusted final dose are performed by distinct algorithms.

11. The METHOD (1000) according to claim 1, wherein the at least one initial user is a plurality of initial users, wherein the at least one final dose comprises a set of final doses, each final dose being associated with a respective initial user, the set of final doses being stored in the database (110).

12. The METHOD (1000) according to claim 11, further comprising selecting a final dose from the set of final doses most compatible with the parameters of the target user.

13. The METHOD (1000) according to claim 11, wherein the adjusted final dose is added to the set of final doses.

14. The METHOD (1000) according to claim 1, further comprising a step of sending the adjusted final dose to a distribution platform (100) by means of a communication network (200).

15.-24. (canceled)

25. A SYSTEM FOR CALCULATING ENERGY DOSES, comprising:
a processor; and
a database (110) associated with the processor,
wherein the processor is configured to perform the steps of:
receiving (1010) a plurality of parameters from at least one initial user;
retrieving (1020) from database (110) at least one initial dose based on at least one of the parameters of the at least one initial user;
adjusting (1030) the at least one initial dose based on at least one of the parameters of the at least one initial user and a simulation of at least one effect of the initial dose on a standard human organism to generate at least one adjusted initial dose;
receiving (1040) feedback information from the at least one initial user regarding the at least one adjusted initial dose;
calculating (1050) at least one final dose based on feedback information from the at least one initial user and the at least one adjusted initial dose;
receiving (1060) feedback information from at least one target user regarding the effect of the at least one final dose; and
calculating (1070) at least one adjusted final dose based on feedback information from the at least one target user and the at least one final dose;
wherein the at least one initial dose, the at least one adjusted initial dose, the at least one final dose and the at least one adjusted final dose comprise electromagnetic energy with a wavelength between 400 to 1000 nm;
wherein the step of adjusting (1030) the at least one initial dose comprises:
determining a power per unit of area E from the at least one initial dose;
simulating the at least one effect of the initial dose from power per unit of area E on at least one biological tissue m of the standard human organism for a redetermined exposure time period t;
wherein the at least one effect of the initial dose comprises the variation in concentration of at least one biological marker n after exposure of the at least one biological tissue m to power per unit of area E for the predetermined time period t and
adjusting the at least one initial dose based on the simulated variation in concentration of the at least one biological marker n and at least one of the parameters of the at least one initial user,
wherein the simulating variation in the concentration of the at least one biological marker n comprises calculating a stimulus Vₘ received from the at least one biological tissue m during the predetermined exposure time t;
wherein the stimulus Vₘ comprises the volumetric energy density integrated over the predetermined period of time for the at least one biological tissue m, that is,
Vₘ=∫∫₀^{t}EdΩdt;
determining the solution of the matrix equation C=M·V, wherein
$$C = ( \begin{matrix}{C}_{1} \\ ⋮ \\ {C}_{n}\end{matrix} ) ,$$
wherein Cₙ is the variation in the concentration of the at least one biological marker
$$M = ( \begin{matrix}{M}_{11} & … & {M}_{1 m} \\ ⋮ & ⋱ & ⋮ \\ {M}_{n 1} & … & {M}_{nm}\end{matrix} )$$
wherein Mₙₘ is the susceptibility of the at least one biological tissue m to modulate the production of the at least one biological marker n; wherein the susceptibility can be any number between −1 and 1, wherein if it is −1, there is complete inhibition of production of the biological marker n; if it is 0, there is no influence, and if it is 1, there is maximum stimulation of production of the biological marker n; and
$$V = ( \begin{matrix}{V}_{1} \\ ⋮ \\ {V}_{m}\end{matrix} )$$
wherein V=(V₁ . . . Vₘ) are the stimuli received in each biological tissue m from the standard human organism during the predetermined exposure time t; and
summing each variation of biological marker n by tissue Cₙ to determine the total variation ΔSₙ of each biological marker n in the standard human organism.

26. The SYSTEM, according to claim 25, wherein the processor is further configured to send the at least one adjusted initial dose and the at least one final dose to an energy dose application device (20) by means of a communication network (200).

27. The SYSTEM, according to claim 25, wherein the processor is further configured to receive feedback information from the at least one initial user and the at least one target user from an energy dose application device (20) by means of a communication network (200).

28.-36. (canceled)
